(19) United States
(12) Patent Application Publication
(10) Pub. No.: US 2009/0149479 A1
(43) Pub. Date: Jun. 11, 2009

(54) DOSING REGIMEN

(75) Inventors: Scott Jenkins, Downingtown, PA (US); Gary Liversidge, West Chester, PA (US)

Correspondence Address: Fox Rothschild, LLP;Elan Pharma International Limited, 2000 Market Street, Philadelphia, PA, 19103, US

(73) Assignee: Elan Pharma International Limited, Co. Westmeath (IE)

(21) Appl. No.: 12/209,728

(22) Filed: Sep. 12, 2008

Related U.S. Application Data
(63) Continuation-in-part of application No. 10/827,689, filed on Apr. 19, 2004.
Application No. 10/827,689 is a continuation-in-part of application No. 11/372,857, filed on Mar. 10, 2006.
Application No. 11/372,857 is a continuation-in-part of application No. 10/827,689, filed on Apr. 19, 2004.
Application No. 10/827,689 is a continuation of application No. 10/354,483, filed on Jan. 30, 2003, now Pat. No. 6,793,936.
Application No. 10/354,483 is a continuation of application No. 10/331,754, filed on Dec. 30, 2002, now Pat. No. 6,902,742.
Application No. 10/331,754 is a continuation of application No. 09/850,425, filed on May 7, 2001, now Pat. No. 6,730,325.
Application No. 09/850,425 is a continuation of application No. 09/566,636, filed on May 8, 2000, now Pat. No. 6,228,398.
Application No. 09/566,636 is a continuation of application No. PCT/US99/25632, filed on Nov. 1, 1999.
(60) Provisional application No. 60/971,882, filed on Sep. 12, 2007.
(60) Provisional application No. 60/106,726, filed on Nov. 2, 1998.

(51) Int. Cl. A61K 31/52

(52) U.S. Cl. 514/263.4

(57) ABSTRACT

Methods and kits are provided enabling a twice daily dosing regimen that achieves daily patient blood levels of active pharmaceutical ingredient comparable to a dosing regimen requiring the same active ingredient to be administered three times a day.

### RELATIONSHIP TO PRIOR APPLICATIONS

This application claims the benefit of priority from U.S. Provisional Application No. 60/971,882 filed on Sep. 12, 2007. This application is also a continuation in part of U.S. application Ser. No. 10/827,689 filed on Apr. 19, 2004. This application is also a continuation in part of U.S. application Ser. No. 11/372,857 filed on Mar. 10, 2006, which is a continuation-in-part of application Ser. No. 10/827,689, filed Apr. 19, 2004, which is a continuation of application Ser. No. 10/354,483, filed Jan. 30, 2003, now U.S. Pat. No. 6,793,936, which in turn is a continuation of application Ser. No. 10/331,754, filed Dec. 30, 2002, now U.S. Pat. No. 6,902,742, which in turn is a continuation of application Ser. No. 09/850,425, filed May 7, 2001, now U.S. Pat. No. 6,730,325, which in turn is a continuation of application Ser. No. 09/566,636, filed May 8, 2000, now U.S. Pat. No. 6,228,398, which in turn is a continuation of Application No. PCT/US99/25632, filed Nov. 1, 1999, which claims the benefit of provisional Application No. 60/106,726, filed Nov. 2, 1998. The disclosure of all these applications is incorporated herein by reference in its entirety.

### FIELD OF THE INVENTION

The present invention relates generally to methods of drug administration; in particular, to methods and kits enabling twice daily administration of a drug that provides daily patient blood levels of the drug comparable to that achieved by a dosing regimen requiting the same drug to be administered three times a day.

### BACKGROUND OF THE INVENTION

The following discussion of the background of the invention is merely provided to aid the reader in understanding the invention and is not admitted to describe or constitute prior art to the invention.

Dosing regimens requiting a large number of doses to be taken daily, for example, three times a day, are difficult for many patients to comply with. For example, three times a day dosing may be may be a challenging dosing regimen for pediatric patients, particularly in cases in which schools will not administer drug products to children during the school day. For senior citizens and others who must adhere to complicated schedules involving a number of drugs to be taken several times during the day, compliance may also be difficult. Compliance rates may be improved by requiring less frequent administration of the drug(s). However, certain drugs that are typically dosed three times a day may be difficult to formulate as products to be administered once daily or twice daily, since this may require significant clinical and safety testing, due to, for example, different delivery and/or pharmacokinetic profiles of the formulations.

In addition, drugs that are administered in combination may have differing delivery and/or pharmacokinetic profiles, leading to periods in which the level of one of the drugs is sub-therapeutic. For certain indications, it may be desirable to have a drug-free period with respect to one of the drugs. In such cases, the dosing regimen may become quite complicated, leading to low compliance by patients.

Therefore, there remains a need for methods and combinations that enable the twice daily dosing of a drug that mimics the effects of three times daily dosing of the same drug.

### BRIEF SUMMARY OF THE INVENTION

In certain aspects, the present invention relates to a drug dosing regimen requiring fewer doses per day than the conventional dosage regime. Certain aspects of the invention provide combinations of immediate release (IR) and delayed release (DR) drug formulations that achieve blood levels of the active pharmaceutical ingredient that mimic the blood levels of the active agent administered via a dosing regimen requiring a greater number of doses per day. Aspects of the invention include using combinations of IR and DR dosage forms in a twice a day dosing regimen which achieves daily patient blood levels comparable to that achieved with three times a day dosing of the same active agent.

Certain aspects of the invention relate to a method of presenting a twice a day course of drug treatment to a patient comprising (A) presenting to a patient, on a daily basis, a first daily dose comprising at least one dosage form comprising an active drug agent, wherein said first dose comprises an immediate release component and a delayed release (DR) component and B) presenting to the patient a second dose comprising an immediate release dosage form of the active agent; such that, upon administration to the patient of the first daily dose at Time 0 and the second daily dose at some time subsequent to Time 0, the pharmacokinetic profile of the active agent in the blood of the patient over a 24-hour period mimics that of administration of the active agent to the patient three times a day.

Certain aspects of the invention relate to a kit for presenting a course of drug treatment to a patient. The kit may comprise (A) at least one first daily dose of an active agent comprising at an immediate release component and a delayed release component; and (B) at least one second daily dose of an active drug agent comprising an immediate release dosage form.

Upon administration to the patient of the first daily dose at Time 0 and the second daily dose at a time subsequent to Time 0, the pharmacokinetic profile of the active agent in the blood of the patient over a 24-hour period mimics that of administration of the active agent to the patient three times a day.

Additional aspects of the invention relate to a twice daily method of administering an active pharmaceutical ingredient to a subject in need thereof comprising the steps of: (A) administering to the subject at Time 0 a first dose comprising at least one dosage form comprising an active agent; the first dose comprises an immediate release component and a delayed release component; and (B) administering to the patient at a time subsequent to Time 0 at least one second dose comprising an immediate release dosage form comprising the active agent, such that the pharmacokinetic profile of the active agent in the blood of the patient over a 24-hour period mimics that of administration of the active agent to the patient three times a day.

According to another aspect, the invention relates to a method of administering a combination of active ingredients to a subject in need thereof where each drug in the combination presents different pharmacokinetic profiles. According to this embodiment, the drugs are presented to a subject in need thereof such that the pharmacokinetic profile of one drug complements, acts in concert, overlaps, or competes in some manner with the other drug. According to an exemplary embodiment, a dosing card that presents the combination of drugs may be used to ensure patient compliance such that both drugs provide coverage over the dosing period.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 represents a dosage card according to an exemplary embodiment of the present invention.

### DETAILED DESCRIPTION OF THE INVENTION

### Definitions

As employed above and throughout the disclosure, the following terms, unless otherwise indicated, shall be understood to have the following meanings.

As used herein, “about” will be understood by persons of ordinary skill in the art and will vary to some extent according to the context in which it is used. If there are uses of the term which are not clear to persons of ordinary skill in the art given the context in which it is used, “about” will mean up to plus or minus 10% of the particular term.

As used herein, “therapeutically effective amount” means the dosage that provides the specific pharmacological response for which the active agent is administered in a significant number of subjects in need of the relevant treatment. It is emphasized that a therapeutically effective amount of the active agent that is administered to a particular subject in a particular instance will not always be effective in treating the conditions described herein, even though such dosage is deemed to be a therapeutically effective amount by those of skill in the art.

As used herein, the term “subject” is used to mean an animal, preferably a mammal, including a human. The terms “patient” and “subject” may be used interchangeably. Thus, certain embodiments of the invention are directed to appropriate dosage forms useful in the administration of active pharmaceutical ingredients to a subject.

As used herein, the term “active pharmaceutical ingredient” or “active agent” includes active drug substances described herein or known in the art, as well as salts, derivatives, conjugates, hydrates, polymorphs, and analogues thereof. The active agent may be present either in the form of one substantially optically pure enantiomer or as a mixture, racemic or otherwise, of enantiomers.

As used herein, the term “IR” describes an active ingredient delivered to a subject in need thereof in an immediate release fashion as would be understood by one of ordinary skill in the art.

As used herein, the term “DR” describes an active ingredient delivered to a subject in need thereof in a manner other than in an IR fashion as defined herein and includes delayed release of an active pharmaceutical ingredient. Such a DR delivery manner includes delivery mechanisms as would be known in the art including: CR—controlled release, ER—extended release, MR— modulated release; pulsed release, and zero order release.

According to one embodiment, the invention is directed to a once a day administration of a drug that provides daily patient blood levels of the drug comparable to that achieved by a dosing regimen requiring the same drug to be administered twice a day, three times a day, four times a day, or more. According to another embodiment, the invention is directed to twice daily administration of a drug that provides daily patient blood levels of the drug comparable to that achieved by a dosing regimen requiring the same drug to be administered three times a day, four times a day, or more.

In certain aspects, the invention relates to combinations of IR and DR drug formulations that provide blood levels of the active agent that mimic the blood levels of the drug administered via a dosing regimen requiring a greater number of doses per day. In certain embodiments, twice daily dosing using IR and DR formulations provides blood levels of active agent over a 24-hour period that are comparable to that achieved by a dosing regimen requiring administration three times a day. In certain preferred embodiments, the dosage form provides a delayed release of the drug. In certain aspects, the drug formulated in the IR and DR formulations is the same drug, or a different drug as described in the combination therapy aspect of the present invention.

In certain aspects of the invention, twice daily administration of a drug formulation to mimic thrice daily dosing may be achieved by administering (at Time 0) a first dose comprising a drug formulation that contains an IR component and a DR component comprising the active agent, coupled with administration, at a subsequent time, of a second dose comprising an immediate release component comprising the active agent. Preferably, the first dose is administered in the morning and the second dose is administered later in the day, preferably close to bedtime. In certain preferred embodiments, administration of the second daily dose takes place about 12 to about 20, about 14 to about 18, or about 16 hours after the administration of the first dose at Time 0. In certain embodiments, the first dose may comprise a delayed release component that allows for release of the active agent subsequent to the release of the IR component. In a preferred embodiment, the delayed release component allows for release of the active agent approximately eight hours after administration.

In certain embodiments of the invention, the IR and DR formulations that comprise the first daily (a.m.) dose administered at Time 0 are combined in the same dosage form.

In another embodiment of the invention, the combination of immediate release and delayed release active agent administered at Time 0 may be achieved by the co-administration of two separate formulations; one in a dosage form providing immediate release of the active agent, and the other in a dosage form providing a delayed release of the active agent.

In one embodiment, the IR and DR formulations are presented to the patient in a package having an a.m. and a p.m. section for each day of a specified treatment period. In one embodiment, the a.m. section of the package comprises a formulation comprising an IR component and a DR component in combination, while the p.m. section of the package comprises an IR dosage form of the drug.

In another embodiment, the IR formulation and the DR formulation may be each be contained in a separate dosage form that is packaged together in the a.m. section of the packaging for administration of the first dose. The second dose in the p.m. section comprises an IR dosage form.

In certain embodiments, the packaging used to present the IR and DR formulations to the patient may be in the form of dosing cards, pill packs, or any other suitable packaging forms.

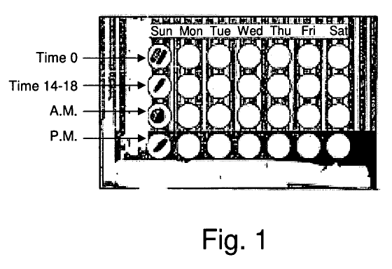

In certain preferred embodiments, the packaging is in the form of a dosing card. Preferably, the dosing card comprises an a.m. section presenting the formulation or formulations to be administered at Time 0, as well as a p.m. section presenting the formulation to be administered at a time subsequent to Time 0 as is illustrated in FIG. 1.

In other embodiments, the IR formulation and the DR formulation may be each be contained in the same dosage form and presented to the patient in a discrete package form, e.g., a first bottle. A second package or bottle may also be given to the patient. The second bottle contains only the IR formulation. According to an embodiment, the patient in need of the therapeutic effect of the active ingredient formulated in the IR and DR formulations is instructed to take the IR/DR single dosage form from the first package in the am, and instructed to take the IR dosage form from the second package in the pm. The formulation in combination with the prescribing regime decreases the number of dosage forms that a patient would be required to ingest compared to conventional therapies of the same active ingredient.

In other aspects of the invention, combinations of active agents may be administered. In one embodiment, a combination of drugs differing in pharmacokinetic profiles may be presented to a patient such that the morning dose (Time 0) comprises an IR component comprising a long-acting agent, and an IR component comprising a short-acting agent. The p.m. dose comprises an IR component comprising the short-acting agent. In certain preferred embodiments, the long-acting agent may have a duration of action of about 24 hours and the short-acting agent may have a duration of action of about 12 hours. This dosing regimen provides for twice daily dosing to maintain efficacy of both active agent 24 hours a day.

In other aspects of the invention, it may be desirable to administer a combination of active agents to allow for a drug-free period of time with respect to one of the active agents. In one embodiment, a dose is administered in the morning (Time 0) comprising an IR component comprising a longer-acting agent and an IR component comprising a shorter-acting agent. The p.m. dose comprises an IR dosage form comprising the shorter-acting drug. In certain preferred embodiments, the longer-acting agent may have a duration of action of about 18 hours, while the shorter-acting agent may have a duration of about 12 hours. This dosing regimen provides for 24-hour efficacy of the longer-acting agent, while providing that the shorter-acting agent is present only during the day.

In certain other aspects, the invention relates to combination therapies where it is desirable to have complementary, overlapping, or even competing pharmacokinetic profiles of at least two, and possibly three or more, active ingredients.

One example of the combination therapy aspect of the invention is the combination of at least two active ingredients such as the combination of SINGULAIR® (montelukast sodium tablets with 24 hour duration manufactured by Merck & Co., Inc., of Whitehouse Station, N.J.) in co-treatment, co-administration, or co-formulated with ALLEGRA-D® (fexofenadine HCl extended release tablets with 12 hour duration manufactured by Sanofi-Aventis of Bridgewater, N.J.). In this example, the patient requires efficacious levels of both the 24 hour montelukast and 12 hour fexofenadine to treat rhinitis over a total of 24 hours. According to an exemplary embodiment, a product that includes the combination of montelukast and fexofenadine may be formulated into one dosage form or alternatively packaged in a dosing card and prescribed in such a manner as to allow for twice daily dosing to maintain efficacy of each active ingredient over the 24 hour dosing period.

In other exemplary embodiments, two active agents may be used. For example, in a set of embodiments, the first active ingredient is administered to treat a medical condition, but this first agent has undesirable side effects. To manage the side effects of the first agent, the second agent is provided within the kit.

For example, a combination of an opiate and a stimulant can be used. Opiates are widely used for pain management, but the side effects of opiate treatment regiment include fatigue and sleepiness. These side effects may be overcome if opiate treatment regiment is complemented with stimulant treatment. The kits of the instant invention may be designed in such a way to ensure active effect of stimulants during the day time, while the effect of an opiate would last twenty-four hours. For example, the stimulant may be combined within the delayed release component of the first daily dose of the opiate.

In another non-limiting example, a combination of an opiate and a laxative is provided. As known in the art, an administration of an opiate may cause constipation, which may be severe. To manage the constipation, a laxative is provided. In one embodiment, a laxative is provided in the IR component such that it exerts its effect only for a relatively short period.

Other suitable combinations of agents include a chemotherapeutic agent for cancer treatment, and a second active agent which decreases undesirable side effects of the chemotherapeutic agent. The side effects of chemotherapeutic agents include, without limitations, fatigue, hair loss, nausea and/or vomiting, anemias and infections. It is important to keep in mind, however, that not every person gets every side effect. Accordingly the choice of the suitable second agent depends on the nature of the side effects in a particular patient treated with the chemotherapeutic agent. In some embodiments of the invention (for example, wherein the side effect is anemia), the second active agent would be present in the IR and DR components of the first dose as well as in the second dose.

Exemplary active ingredients include but are not limited to drug compounds acting on the central nervous system such as psychostimulants and cerebral stimulants, for example methylphenidate; aldosterone inhibitors such as spironolactone, eplernone and analogs thereof; alkaloids; alpha/beta-blockers such as labetalol, carvedilol and analogs thereof; analgesics such as acetaminophen, naproxen, paverin, and analogues thereof tramadol and opioids such as morphine, codeine, thebaine, heroin, oxycodone, hydrocodone, dihydrocodeine, hydromorphone, oxymorphone, buprenorphine, etorphine, naloxone, nicomorphine, methadone, pethidine, fentanyl, alfentanil, sulfentanil, remifentanil, carfentanyl, pentazocine, phanazocine, butorphanol, levorphanol and analogs thereof; anesthetics such as lidocaine and bupivacaine and analogs thereof; anorectics such as benzphetamine, diethylpropion, mazindol, phendimetrazine, and phentermine; anti-adrenergic agents such as centrally and peripherally acting anti-adrenergic agents and analogs thereof; anti-allergic agents; anti-anginal agents such as nitroglycerine and analogs thereof; anti-arrhythmic agents such as moricizine, ibutilide, quinidine, procainamide, diisopyramide, lidocaine, tocainide, flecainide, mexiletine, propafenone, bretylium, amiodarone, adenosine, dofetilide and analogs thereof; anti-asthmatic agents such as salbutamol and analogs thereof; antibiotics such as aminosalicylic acid, amoxicillin, amoxicillin and potassium clavulanate, ampicillin, ampicillin and sulbactam, azithromycin, bacampicillin, carbenicillin, carbenicillin indanyl sodium, capreomycin, cefadroxil, cefazolin, cefcapene pivoxil, cephalexin, cephalothin, cephapirin, cephacelor, cefprozil, cephadrine, cefamandole, cefonicide, ceforanide, cefuroxime, cefixime, cefoperazone, cefotaxime, cefpodoxime, ceftaxidime, ceftibuten, ceftizoxime, ceftriaxone, cefepime, cefinetazole, cefotetan, cefoxitin, ciprofloxacine, clarithromycin, clindamycin, clofazimine, cloxacillin, cotriamoxazole, cycloserine, dicloxacillin, dirithromycin, erythromycin, ethambutol, ethionamide, fosfomycin, imipenem, isoniazide, levofloxacine, lomefloxacine, loracarbef, methicillin, methenamine, metronidazole, metoclopramide, meziocillin, nafcillin, nalidixic acid, nitrofurantoin, norfloxacin, novobiocin, ofloxacin, oxacillin, penicillin, pentamidine, piperacillin, piperacillin and tazobactam, sparfloxacin, sulphacytine, sulphamerazine, sulphamethazine, sulphamethixole, sulphasalazine, sulphisoxazole, sulphapyrizine, sulphadiazine, sulphmethoxazole, sulphapyridine, ticarcillin, ticarcillin and potassium clavulanate, trimethoprime, trimetrexate, troleanomycin, vancomycin, verapamil and analogs thereof; anti-cancer agents; anti-coagulant agents such as heparin, hirudin and analogs thereof; haemostatic agents; anti-convulsants such as carbamazepine, levetiracetam, topiramate and analogs thereof; anti-depressant agents such as amitriptyline, amoxapine, bupropion, citalopram, clomipramine, desipramine, doxepin, escitalopram, fluoxetine, fluvoxamine, imipramine, maprotiline, mirtazapine, nefazodone, nortriptyline, paroxetine, phenelzine, protriptyline, sertraline, tranylcypromine, trazodone, trimipramine, venlafaxine, and analogs thereof; anti-diabetic agents; anti-diarrheal agents such as loperamide and analogs thereof; anti-emetic agents such as scopolamine, ondensetron, domperidone, metoclopramide and analogs thereof; anti-epileptic agents; anti-fungal agents such as acylanilide and analogs thereof; antihistamines such as terfenadine and analogs thereof; anti-hypertensive agents, such as atorvastatin, rosuvastatin, lovastatin, etc.; anthelmintics; anti-inflammatory agents; anti-migraine agents such as sumatriptan, ergot alkaloids and analogs thereof; antimuscarinic agents; antimycobacterial agents; anti-neoplastics such as fluorouracil, bleomycin and analogs thereof; anti-parkinsonian agents; anti-psychotic agents such as acetophenazine, aripiprazole, chlorprothixene, droperidol, olanzapine, promazine, quetiapene, risperidone, sulpiride, trifluopromazine, ziprasidone, and analogs thereof; anti-rheumatic agents such as fentiazac and analogs thereof; anti-thrombic agents; anti-tussive agents; anti-ulcer agents such as 5-asa, cimetidine, famotidine, lansoprazole, omeprazole, ranitidine and analogs thereof; anti-viral agents such as acyclovir, famciclovir, ganciclovir, zidovudine and analogs thereof; anxiolytic agents such as alprazolam, buspirone, clonazepam, clorazepate, chlordiazepoxide, diazepam, hydroxyzine, lorazepam, meprobamate, oxazepam, and analogs thereof; ARB blockers, such as irbesartan, candesartan, losartan, valsartan, telmisartan, eprosartan and analogs thereof; beta-blockers, such as acebutolol, atenolol, betaxolol, bisoprolol, esmolol, metoprolol, carteolol, nadolol, penbutolol, pindolol, propranolol, sotalol, timolol, labetalol and analogs thereof; blood lipid-lowering agents such statins such as simvastatin and analogs thereof; calcium channel blockers such as nifedipine, verapamil, diltiazem, nicardipine, nisoldipine, nimodipine, isradipine, bepridil, felodipine, amlodipine and analogs thereof; cardiovascular agents, anti-hypertensive agents and vasodilators such as benazepril, captopril, clonidine, enalapril, fosinopril, isosorbide dinitrate, isosorbide-5-mononitrate, hydralizine, lisinopril, moexipril, pentoxifylline, perindopril, prazosine, quinapril, quinidine, ramipril, trandolapril, nitrates, peripheral vasodilators and analogs thereof; chelating agents such as deferoxamine and analogs thereof; chemotherapy agents such as vincristine and analogs thereof; contraceptives; diuretic agents such as loop diuretics, acetazolamide, amiloride, bendroflumethiazide, bumetamide, chlorthalidone, chlorothiazide, dichlorphenamide, ethacrynic acid, furosemide, hydrochlorothiazide, hydroflumethiazide, indapamide, mannitol, methazolamide, methylclothiazide, metolazone, naturetin, polythiazide, spironolactone, triameterene, triamterene, trichloromethiazide, triamterene, torsemide, and analogs thereof; anti-diuretics; fertility promoters; dopaminergic agents; hypnotic agents such as amobarbital, butabarbital, chloral hydrate, estazolam, flurazepam, mephobarbital, paraldehyde, pentobarbital, phenobarbital, quazepam, secobarbital, temazepam, triazolam, zaleplon, zolpidem and analogs thereof; inducers and inhibitors of uterine labor; immunosuppressants; inotropic agents such as digoxin and analogs thereof; narcotic antagonists; NSAIDs such as celecoxib, etoricoxib, rofecoxib, valdecoxib, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, tiaprofenic acid, salicylates such as acetylsalicylic acid, choline magnesium salicylate, choline salicylate, magnesium salicylate, and sodium salicylate, and analogs thereof; anti-thyroid agents; astringents; neuroleptic agents; synthetic and naturally occurring peptides, proteins or hormones such as desmopressin, vasopressin, insulin, calcitonin, calcitonin gene regulating protein, atrial natriuretic protein, colony stimulating factor, betaseron, erythropoietin (EPO), interferons such as □, □, □ interferon, somatropin, somatotropin, somatostatin, insulin-like growth factor (somatomedins), luteinizing hormone releasing hormone (LHRH), tissue plasminogen activator (TPA), growth hormone releasing hormone (GHRH), oxytocin, estradiol, growth hormones, leuprolide acetate, factor VIII, interleukins such as interleukin-2 and analogs thereof; prostaglandins and analogs thereof; sedatives such as benzodiazepines, phenothiazines and analogs thereof; and vasoprotective agents. A more detailed description of these classes of drugs and a listing of species within each class can be found in Martindale, The Extra Pharmacopoeia, Twenty-ninth Edition, The Pharmaceutical Press, London, 1989, the disclosure of which is hereby incorporated by reference in its entirety. The active agents are commercially available and/or can be prepared by techniques known in the art.

In certain embodiments of the invention, including, without limitation, aspects in which twice a day administration mimics the effects of three times a day dosing, preferred active agents include, but are not limited to, famciclovir, pregabalin, sevelamer, nelfinavir, indinavir, pramipexole, metaxalone, trientine, thioridazine, rifaximin, alprazolam, carbidopa, levodopa, erythromycin ethylsuccinate, phenyloin, acamprosate, praziquantel, molindone, levocamitine, dichlorphenamide, posaconazole, promethazine, delavirdine, and sildenafil.

Advantages of the methods and kits described herein include reducing the dosing frequency required while still maintaining the benefits normally derived from the conventional dosing regimen. This reduced dosing frequency is particularly advantageous in the case of children in that it eliminates the need for dosing during the middle of the school day which can be both disruptive and embarrassing for the patient. It is also advantageous in terms of patient compliance to have a formulation which may be administered at reduced frequency. The reduction in dosage frequency made possible by utilizing embodiments described herein may contribute to reducing health care costs by reducing the amount of time spent by health care workers on the administration of drugs.

The active agents employed herein may be formulated into any suitable dosage form(s) providing the required combination of immediate release and delayed release, as well as the required immediate release component. In certain aspects, the IR and DR form may be combined in the same dosage form. Alternatively, separate dosage forms may provide the IR and DR components and may, in certain aspects, be combined by packaging for presentation to the patient, providing convenience to the patient and providing for ease of compliance.

Immediate release components and dosage forms employed herein may be made by any suitable methods known to the art.

Release of certain active agents, including, without limitation, delayed release, may be achieved by any suitable means known to the art. Examples include, without limitation, U.S. Pat. No. 6,228,398 to Devane et al., titled “Multiparticulate Modified Release Composition”, hereby incorporated by reference.

Any coating material which modifies the release of the active ingredient in the desired manner may be used. In particular, coating materials suitable for use in the practice of the invention include, but are not limited to, polymer coating materials, such as cellulose acetate phthalate, cellulose acetate trimaletate, hydroxy propyl methylcellulose phthalate, polyvinyl acetate phthalate, ammonio methacrylate copolymers such as those sold under the Trade Mark EUDRAGIT® RS and RL, and other polymer coating materials as would be understood by one of ordinary skill in the art.

In certain embodiments, delayed release may be achieved by a release component comprising matrix material. When the delayed release component comprises a matrix material, any suitable matrix material or suitable combination of matrix materials may be used. Such materials are known to those skilled in the art and include, but are not limited to, hydrophilic polymers, hydrophobic polymers and mixtures thereof which are capable of modifying the release of an active ingredient dispersed therein in vitro or in vivo. More specific matrix materials suitable for the practice of the present invention include, but are not limited to, microcrystalline cellulose, sodium carboxymethylcellulose, hydroxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate and mixture thereof.

In certain embodiments, active agents may be incorporated into a suitable dosage form which facilitates release of the active ingredient in a pulsatile manner. Typically, the dosage form may be a blend of the different populations of active ingredient containing particles which make up the immediate release and the delayed release components, the blend being filled into suitable capsules, such as hard or soft gelatin capsules. Alternatively, the different individual populations of active ingredient containing particles may be compressed (optionally with additional excipients) into mini-tablets which may be subsequently filled into capsules in the appropriate proportions. Another suitable dosage form is that of a multilayer tablet. In this instance the first component of the multiparticulate composition may be compressed into one layer, with the second component being subsequently added as a second layer of the multilayer tablet. The populations of active ingredient-containing particles making up the composition may further be included in rapidly dissolving dosage forms such as an effervescent dosage form or a fast-melt dosage form.

In certain embodiments, compositions are employed that comprise at least two populations of active ingredient-containing particles which have different in vitro dissolution profiles.

Preferably, solid oral dosage forms are employed. Solid dosage forms for oral administration include, but are not limited to, tablets, capsules, sachets, lozenges, powders, pills, or granules, and the solid dosage form can be, for example, a fast melt dosage form, lyophilized dosage form, or a combination thereof.

In general, the dosage forms employed will be administered to a subject in need thereof using a level of a drug or an active agent that is sufficient to provide the desired physiological effect. The effective amounts of the active agent of the compositions of the invention can be determined empirically and can be employed in pure form or, where such forms exist, in pharmaceutically acceptable salt, ester, or prodrug form. Actual dosage levels of the active agent in the dosage form may be varied to obtain an amount of the active agent that is effective to obtain a desired therapeutic response for a particular composition and method of administration and the condition to be treated. The selected dosage level therefore depends upon the desired therapeutic effect, the route of administration, the potency of the administered active agent, the desired duration of treatment, and other factors. The level of active agent needed to give the desired physiological result is readily determined by one of ordinary skill in the art by referring to standard texts, such as Goodman and Gillman and the Physician's Desk Reference.

Dosage unit compositions may contain such amounts of such submultiples thereof as may be used to make up the daily dose. It will be understood, however, that the specific dose level for any particular subject will depend upon a variety of factors: the type and degree of the cellular or physiological response to be achieved; activity of the specific agent or composition employed; the specific agent(s) or composition employed; the age, body weight, general health, sex, and diet of the patient; the time of administration, route of administration, and rate of excretion of the active agent; the duration of the treatment; active agents used in combination or coincidental with the specific active agent; and like factors well known in the medical arts.

In certain embodiments, the solid oral dosage forms release the active ingredient such that substantially all of the active ingredient contained in the first component is released prior to release of the active ingredient from the second component. When the first component comprises an IR and DR combination (either in the same dosage form or separate dosage forms), it is preferable that the IR component releases the active ingredient immediately after ingestion or after placed in the mouth (depending on the dosage form, i.e., fast melt), and the release of the active ingredient in the DR component is delayed until substantially all the active ingredient in the IR component has been released. Release of the active ingredient from the DR component may be delayed as detailed above by the use of release coatings and/or release matrix materials.

Administration of Compositions

In certain embodiments, the present invention also provides methods comprising the administration to a subject in need thereof an effective amount of at least one active agent.

In certain embodiments, a method of administering an active agent to a subject in need thereof comprises the steps of: (A) administering to the subject at Time 0 a first dose comprising at least one dosage form comprising the active agent, wherein the dosage form comprises an IR component and a DR component; and (B) administering to the patient at some time subsequent to Time 0 at least one second dose comprising an immediate release dosage form of the active agent; wherein the pharmacokinetic profile of the active agent in the blood of the patient over a 24-hour period mimics that of administration of the active agent to the patient three times a day. In preferred embodiments of the invention, the second dose is administered to the subject about 12 to about 20 hours after Time 0, about 14 to about 18 hours after Time 0, or about 16 hours after Time 0.

Certain aspects of the invention may be directed to the administration to a subject of a therapeutically effective amount of a composition comprising an anti-viral agent. In a preferred embodiment, famciclovir is an active agent administered according to various aspects of the invention.

### EXAMPLES

### Example 1

A twice daily dosing regimen is provided for the treatment of Herpes Zoster, for which the current recommended dosage is 500 mg of famciclovir every 8 hours (i.e., three times a day) for 7 days. A dosing card as described herein is provided that contains two types of doses: one to be taken after waking in the morning (Time 0), the other before bedtime (Time 12-18 h). A seven day dosing regimen is provided on the card.

The morning dose (Time 0) consists of two oral dosage forms (such as tablets or capsules) each containing 500 mg of famciclovir, with 250 mg formulated for immediate release and 250 mg formulated for a delayed release of 8 hours.

The bedtime dose (Time 12-18 h) is formulated as 500 mg of immediate release famciclovir.

Administration using the above twice daily dosing regimen mimics the pharmacokinetics of the conventional (three times daily) dosing regimen.

The clinical benefits are expected to be increased patient compliance by simplifying the dosing regimen from three times a day to twice a day.

### Example 2

A dosing card is provided as in Example 1 above, except that each morning dose (Time 0) comprises two separate oral dosage forms (which may be tablets or capsules) provided together in the a.m. section of the card; the first dosage form contains 500 mg of famciclovir formulated for immediate release and the second contains 500 mg of famciclovir formulated for a delayed release of 8 hours.

### Example 3

An example of a long-acting agent that may be paired with a short-acting agent includes montekulast, having a 24-hour duration of action, paired with fexofenadine, which has a 12-hour duration of action. These agents are formulated and packaged in a dosing card having a breakfast (a.m.) and dinner (p.m.) section each day of a specified treatment period. Administration according to the dosing schedule allows for twice-daily administration to maintain efficacy over a 24-hour period for the treatment of rhinitis. This is achieved by dosing in the morning with a combination of an IR montekulast element and an IR fexofenadine element, and dosing at dinnertime with an IR element of fexofenadine.

### Example 4

An additional example is controlled-release 12-hour oxycodone (e.g., OXYCONTIN®, Purdue Pharma) co-administered with modafinil, which has a duration of about 18 hours. Co-administration as described herein may provide continual levels of oxycodone to treat pain but allow for modafinil to be present only during the day to overcome somnolence, an adverse side effect of oxycodone.

Oxycodone is a short acting drug and it is desirable to have a drug-free period between presentations of the drug to a patient. Such a drug-free period is achieved by dosing, in the a.m., an IR/DR formulation comprising oxycodone in the in a controlled released formulation in combination with the longer-acting modafinil. The p.m. IR component taken by the patient at dinnertime would include only the oxycodone controlled release formulation. The treatment regime may be presented on a dosing card with a breakfast (a.m.) and dinner (p.m.) section for each day of a specified treatment period. A patient following such a treatment regime that includes the IR/DR and IR compositions benefits from a reduction in the number of times that a dosage form is required to be taken; thereby, statistically, increasing compliance.

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

The terms and expressions which have been employed are used as terms of descriptions and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope on this invention.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

The disclosures of each patent, patent application and publication cited or described in this document are hereby incorporated herein by reference, in their entirety.

What is claimed is:

1. A method of presenting a course of drug treatment comprising the steps of:
(A) presenting to a patient at least one first daily dose comprising at least one dosage form comprising an active agent, wherein said first dose comprises an immediate release component and a delayed release component;
(B) presenting to the patient at least one second dose comprising an immediate release dosage form of said active agent;
wherein, upon administration to said patient of the first daily dose at Time 0 and the second daily dose at a time about 12 to about 20 hours after Time 0, the pharmacokinetic profile of the active agent in the blood of the patient over a 24-hour period mimics that of administration of the active agent to the patient three times a day.

2. The method of claim 1, wherein in (A), the at least one first daily dose comprises
two dosage forms; the first dosage form comprising an immediate release component, and the second dosage form comprising a delayed release component.

3. A kit for presenting a course of drug treatment to a patient comprising:
(A) at least one first daily dose of an active agent comprising at least one dosage form comprising an immediate release component and a delayed release component;
(B) at least one second daily dose of an active agent comprising at least one immediate release dosage form;
wherein, upon administration to said patient of the first daily dose at Time 0 and the second daily dose at a time about 12 to about 20 hours after Time 0, the pharmacokinetic profile of said active agent in the blood of the patient over a 24-hour period mimics that of administration of the active agent to the patient three times a day.

4. The kit of claim 3 wherein the (A) the at least one first daily dose comprises two dosage forms; the first dosage form comprising an immediate release component, and the second dosage form comprising a delayed release component.

5. The kit of claim 3 wherein the kit comprises a dosage card.

6. The kit of claim 3 wherein the active agent is selected from the group consisting of famciclovir, pregabalin, sevelamer, nelfinavir, indinavir, pramipexole, metaxalone, trientine, thioridazine, rifaximin, alprazolam, carbidopa, levodopa, erythromycin ethylsuccinate, phenyloin, acamprosate, praziquantel, molindone, levocarnitine, dichlorphenamide, posaconazole, promethazine, delavirdine, sildenafil and mixtures thereof.

7. The kit of claim 6 wherein the active agent is famciclovir.

8. A method of administering an active agent to a subject in need thereof comprising the steps of:
(A) administering to the subject at Time 0 a first dose comprising at least one dosage form comprising the active agent, wherein the dosage form comprises an immediate release component and a delayed release component;
(B) administering to the patient about 12 to about 20 hours after Time 0 at least one second dose comprising an immediate release dosage form of the active agent;
wherein the pharmacokinetic profile of the active agent in the blood of the patient over a 24-hour period mimics that of administration of the active agent to the patient three times a day.

9. A kit for presenting a course of drug treatment to a patient comprising:
(A) at least one first daily dose of a first active agent comprising at least one dosage form comprising an immediate release component and a delayed release component;
(B) at least one second daily dose of a first active agent comprising at least one immediate release dosage form;
(C) at least one first daily dose of a second active agent;
wherein, upon administration to said patient of the first daily dose at Time 0 and the second daily dose at a time about 12 to about 20 hours after Time 0, the pharmacokinetic profile of said first active agent in the blood of the patient over a 24-hour period mimics that of administration of the first active agent to the patient three times a day, and wherein the kit.

10. The kit of claim 9, wherein
A) said at least one first daily dose of the first active agent and said at least one first daily dose of a second active agent are presented within a single dose; or
B) said at least one second daily dose of the first active agent and said at least one first daily dose of a second active agent are presented within a single dose; or
C) a combination thereof.

11. The kit of claim 10, wherein said at least one first daily dose of the first active agent and said at least one first daily dose of a second active agent are presented within a single dose and wherein further:
A) said at least one first daily dose of a second active agent is presented within the immediate release component; or
B) said at least one first daily dose of a second active agent is presented within the delayed release component; or
C) a combination thereof.

12. The kit of claim 9, wherein the first active agent causes a side effect amenable to treatment with the second active agent.

13. The kit of claim 9, wherein the first active agent is an opiate, and the second active agent is a stimulant or a laxative.

14. The kit of claim 13, wherein the laxative is present in
A) the immediate release component of said at least one first daily dose; or
B) the at least one second daily dose; or
C) a combination thereof.

15. The kit of claim 13, wherein the stimulant is present in the delayed release component of said at least one first daily dose.
